(19) United States
(12) Patent Application Publication
(10) Pub. No.: US 2009/0131837 A1
(43) Pub. Date: May 21, 2009

(54) Ultrasound Device

(75) Inventor: Nicholas Granville, York (GB)

Correspondence Address: DIANA HOUSTON;SMITH & NEPHEW, INC., 1450 BROOKS ROAD, MEMPHIS, TN, 38116, US

(73) Assignee: SMITH & NEPHEW, PLC, London (GB)

(21) Appl. No.: 11/912,382

(22) PCT Filed: Apr. 21, 2006
(86) PCT No.: PCT/GB06/01488

§ 371 Date: Jun. 9, 2008

(30) Foreign Application Priority Data
Apr. 23, 2005 (GB) 0508254.0

(51) Int. Cl. A61N 7/00

(52) U.S. Cl. 601/2

(57) ABSTRACT

A method an apparatus for healing bone fractures comprises applying an ultrasound signal to a target site. The ultrasound signal comprises a generally uniform distribution of constructive interference positions in the target site.

### CROSS-REFERENCE TO RELATED APPLICATIONS

This application is a National State of International Application No. PCT/GB2006/001488, filed Apr. 21, 2006, which claims the benefit of Priority Document No. 0508254.0, filed Apr. 23, 2005. The disclosure of each application is incorporated by reference in its entirety.

### BACKGROUND

This invention relates to the use of ultrasound, particularly for the healing of bone fractures. This invention relates to a method and an apparatus using ultrasound.

Duarte U.S. Pat. No. 4,530,360 describes a technique of treating bone defects, such as bone fractures, non-unions and pseudarthroses and the like, using a pulsed radio-frequency ultrasonic signal applied via a transducer to the skin of a patient and directing sound waves to the bone defect to be healed. The pulsed radio frequency signal has a frequency in the range of 1.3-2 MHz, and consists of pulses generated at a rate in the range 100-1000 Hz, with each pulse having a duration in the range 10-2,000 microseconds. The power intensity of the ultrasound signal is no higher than 100 milliwatts per square centimeter.

Winder U.S. Pat. No. 5,520,612 describes a technique of treating bone fractures using an electric-acoustic transducer for direct application of ultrasound-frequency energy to the skin in which the transducer is excited with a low-frequency modulation of an ultrahigh-frequency carrier. The carrier frequency is in a range between 20 kHz and 10 MHz, and the modulation frequency has a range between about 5 Hz and 10 kHz. The excitation of the transducer is maintained at an intensity for acoustic-energy coupling to body tissue and/or fluids such that the intensity is less than 100 milliwatts per square centimeter at the fracture.

An existing ultrasound device (Exogen) has a waveform that comprises pulses of 1.5 MHz ultrasound, modulated by a 1 kHz wave, and with a duty cycle of 20%. This results in 300 pulses of ultrasound followed by a time period equivalent to 1200 pulses. This will be referred to hereinafter as 300 on pulses followed by 1200 off pulses.

An existing Exogen device comprises a transducer having an intensity, ISA, of 150 mWcm⁻¹. This is the spatial average intensity or the average intensity over the width of the beam. Due to the 20% duty cycle, this leads to a spatial average, temporal average intensity, I_{SATA}, of 30 mWcm⁻². The spatial average intensity is an outcome of the transducer design. The temporal average intensity is a function of the transducer design and the duty cycle. The device transmits pulsed ultrasound so that there is very little chance of the tissue overheating in the region of the fracture. There is evidence to suggest that pulsed ultrasound heals better than continuous wave ultrasound.

The existing Exogen device heals about 80-85% of fractures. This percentage is approximately the same, regardless of which bone is fractured (femur, tibia, etc) and the depth of soft tissue over the fracture site.

It is an aim of the present invention to improve healing of bone fractures by maximising bone repair.

### SUMMARY

According to a first aspect of the present invention, there is provided a method for healing bone fractures, comprising applying an ultrasound signal to a target site, wherein the signal properties are manipulated in order to maximise bone repair.

According to an embodiment of the present invention, a target site is a site where the ultrasound may be applied. A target site may comprise a defect site or sites, such as a bone fracture(s). A target site may comprise soft tissue. A target site may comprise both a defect site(s) and soft tissue.

According to an embodiment of the present invention, maximising bone repair means that the majority, if not all, of the bone affected by the fracture is repaired. It can also mean that the rate of bone repair is increased so that the healing process is accelerated. It can also mean a combination of the above phenomena.

According to an embodiment of the present invention, the ultrasound signal properties are manipulated in order to generate a uniform distribution of constructive interference positions in the target site.

According to an embodiment of the present invention, the ultrasound signal properties are manipulated in order to maximise the density of constructive interference positions in the target site.

Preferably, the ultrasound signal comprises a carrier frequency, a modulation frequency and an intensity.

Preferably, the intensity of the ultrasound at the constructive interference positions is increased without causing overheating.

Preferably, the spatial average intensity of the ultrasound is increased without causing overheating.

Preferably, the ultrasound signal is manipulated by optimising the modulation frequency.

Preferably, the modulation frequency is at least 10 kHz. The modulation frequency may be in the range 10-1000 kHz. The modulation frequency may be in the range 10-500 kHz. The modulation frequency may be in the range 50-400 kHz. The modulation frequency may be in the range 75-350 kHz. The modulation frequency may be in the range 80-300 kHz. The modulation frequency may be in the range 100-300 kHz.

The modulation frequency may affect the distribution of constructive interference. Selecting modulation frequencies in the ranges specified above generates a uniform distribution of constructive interference positions in the target site. Selecting modulation frequencies in the ranges specified above maximises the density of constructive interference positions in the target site.

The modulation frequency may affect the constructive interference distribution, but need not affect the mean energy of the emitted ultrasound. In accordance with some embodiments of this invention, changing the modulation frequency will not change the amount of energy emitted by the transducer, but will change its distribution. Accordingly, potential overheating is prevented.

The carrier frequency may be in the range 20 kHz-10 MHz. The carrier frequency may be in the range 0.1-10 MHz. The carrier frequency may be in the range 1-5 MHz. Preferably, the carrier frequency is in the range 1-3 MHz. More preferably, the carrier frequency is in the range 1-2 MHz. A carrier frequency of about 1.5 MHz is particularly preferred.

The intensity may be in the range 50-1000 mWcm⁻². The intensity may be in the range 50-500 mWcm⁻². The intensity may be in the range 50-300 mWcm⁻². The intensity may be in the range 50-200 mWcm⁻². The intensity may be in the range 100-200 mW cm⁻². Preferably, the intensity is in the range 120-180 mW cm⁻². More preferably, the intensity is in the range 140-160 mW cm⁻². An intensity of 150 mW cm⁻² is particularly preferred.

Preferably, the ultrasound signal is pulsed.

The pulsed ultrasound signal may have a duty cycle in the range 0.1-90%. The duty cycle may be 1-80%. The duty cycle may be 5-60%. The duty cycle may be 5-50%. The duty cycle may be 10-40%. Preferably, the duty cycle is 15-30%. More preferably, the duty cycle is 15-25%. A duty cycle of 20% is particularly preferred.

According to a second aspect of the present invention, there is provided an apparatus for healing bone fractures, comprising: an electro-acoustic transducer for producing an ultrasound signal; and a generator means for exciting the transducer with an electrical-output signal, wherein the apparatus enables manipulation of the ultrasound signal properties in accordance with the first aspect of the present invention.

According to a third aspect of the present invention, there is provided an apparatus for healing bone fractures, comprising: an electro-acoustic transducer for producing an ultrasound signal; and a generator means for exciting the transducer with an electrical-output signal, wherein the ultrasound signal comprises a carrier frequency, a modulation frequency and an intensity.

Preferably, the modulation frequency is optimised.

Preferably, the modulation frequency is at least 10 kHz. The modulation frequency may be in the range 10-1000 kHz. The modulation frequency may be in the range 10-500 kHz. The modulation frequency may be in the range 50-400 kHz. The modulation frequency may be in the range 75-350 kHz. The modulation frequency may be in the range 80-300 kHz. The modulation frequency may be in the range 100-300 kHz.

The carrier frequency may be in the range 20 kHz-10 MHz. The carrier frequency may be in the range 0.1-10 MHz. The carrier frequency may be in the range 1-5 MHz. Preferably, the carrier frequency is in the range 1-3 MHz. More preferably, the carrier frequency is in the range 1-2 MHz. A carrier frequency of about 1.5 MHz is particularly preferred.

The intensity may be in the range 50-1000 mWcm⁻². The intensity may be in the range 50-500 mWcm⁻². The intensity may be in the range 50-300 mWcm⁻². The intensity may be in the range 50-200 mWcm⁻². The intensity may be in the range 100-200 mW cm⁻². Preferably, the intensity is in the range 120-180 mW cm⁻². More preferably, the intensity is in the range 140-160 mW cm⁻². An intensity of 150 mW cm⁻² is particularly preferred.

Preferably, the ultrasound signal is pulsed.

The pulsed ultrasound signal may have a duty cycle in the range 0.1-90%. The duty cycle may be 1-80%. The duty cycle may be 5-60%. The duty cycle may be 5-50%. The duty cycle may be 10-40%.

Preferably, the duty cycle is 15-30%. More preferably, the duty cycle is 15-25%. A duty cycle of 20% is particularly preferred.

### BRIEF DESCRIPTION OF THE DRAWINGS

Reference will now be made, by way of example, to the following drawings, in which:

FIG. 1 shows graphical results for an existing Exogen device;

FIG. 2 shows an enlarged view of part of FIG. 1;

FIG. 3 shows the intensity at the soft-tissue bone interface;

FIG. 4 shows the intensity at the soft-tissue bone interface;

FIG. 5 shows graphical results for a device according to an embodiment of the present invention;

FIG. 6 is an enlarged view of part of FIG. 5;

FIG. 7 shows graphical results for a device according to an embodiment of the present invention;

FIG. 8 is an enlarged view of part of FIG. 7;

FIG. 9 shows the results of a two-dimensional ultrasound model for an existing Exogen device; and

FIG. 10 shows the results of a two-dimensional ultrasound model for a device according to an embodiment of the present invention.

### DETAILED DESCRIPTION

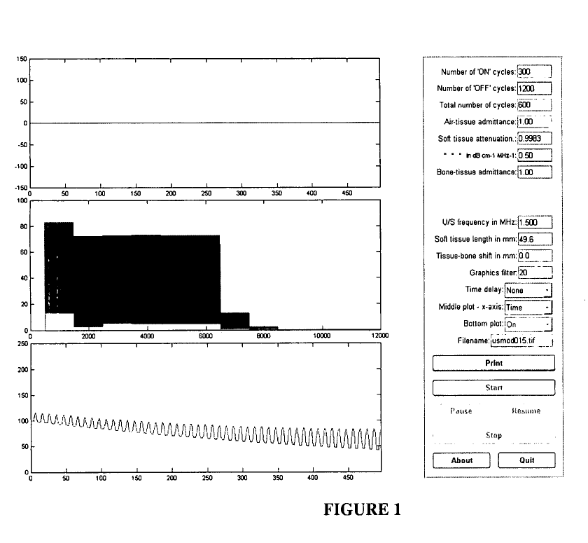

In FIG. 1, the settings that gave rise to the graphical results on the left are shown in the right of the diagram. The first text box shows that there are 300 ‘on’ cycles, which are followed by 1200 ‘off’ cycles (in the second box). The simulation is run for 600 cycles (in the third box). Each cycle is divided into 20 time steps, which is why the central plot has an x-axis that goes up to 12000. The next four boxes set the attenuation and admittance of the ultrasound. The attenuation is 0.5 dB cm⁻¹ MHz⁻¹ (6^{th} box). This equates to 0.9983 per time step (5^{th} box). The admittance at the air-soft tissue and soft tissue-bone interfaces is 1 (4^{th} and 7^{th} boxes), which assumes total reflectance. This represents the worst case scenario. The ultrasound frequency is 1.5 MHz (8^{th} box), and the depth of soft tissue is 49.6 mm (9^{th} box). The remaining text boxes refer to options that are not relevant. This figure shows the ultrasound signal due to the existing Exogen device.

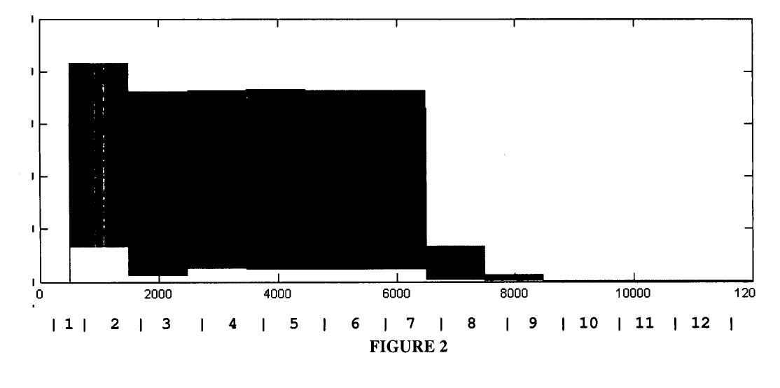

FIG. 2 is an enlarged view of part of FIG. 1. Period 1 is when the ultrasound has started to leave the transducer, but has yet to reach the soft tissue-bone interface. Period 2 is when the ultrasound has reached the interface. Period 3 is when the cycles from period 2 have reached the interface again, and are interfering with new cycles. Periods 4, 5, 6 and 7 are all similar, showing the sum of new cycles plus those from previous periods. Period 8 shows only reflected cycles as the 300 ‘on’ cycles have ended. It is much smaller because of the attenuation occurring going from the transducer to the interface, back to the transducer and then to the interface again. Period 9 shows an even smaller intensity as the ultrasound has traveled between the transducer and the interface five times. It has traveled this distance seven times in period 10, and is now too small to plot. The next series of ‘on’ cycles would start at time step 30500. Clearly, each burst of ultrasound is an independent event. An off period equivalent to 3000 time steps or 150 cycles is sufficient to make each on period an independent event.

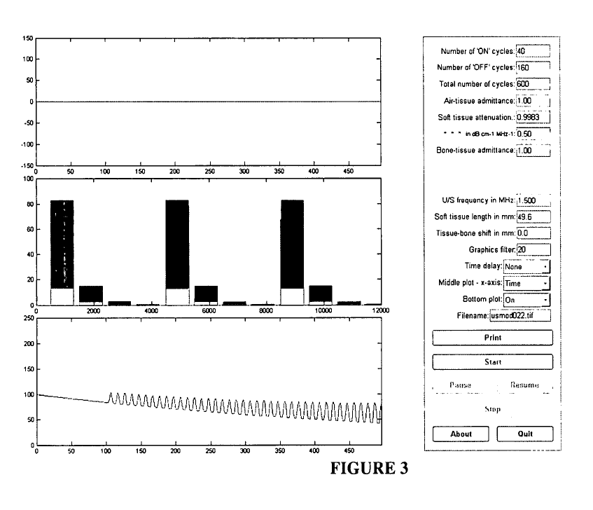

FIG. 3 shows the intensity at the soft-tissue bone interface of 40 ‘on’ cycles followed by 160 ‘off’ cycles. As the duty cycle is the same as the previous example (300/[300+1200]) or (40/[40+160]) or 20%, the energy or mean power of the ultrasound signal is the same.

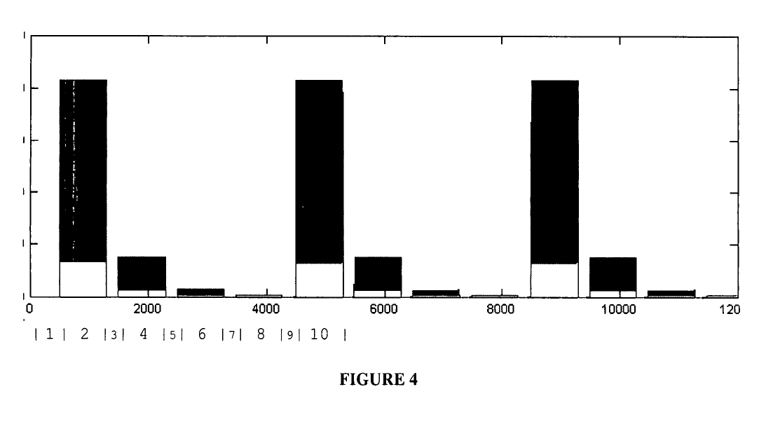

In FIG. 4, periods 1 and 2 are as before, the ultrasound has yet to reach the interface, and the signal reaches the interface. Period 3 is a short period when the ‘on’ cycles have stopped, but the reflected signal has yet to reach the interface. Period 4 shows the reflected signal, attenuated but not showing interference as there are no ‘on’ cycles. Period 5 is another short period between sets of reflected cycles. Period 6 shows a re-reflected signal, and has a lower intensity. The intensity in period 8 can just be shown. Period 10 shows the next set of ‘on’ cycles reaching the soft tissue-bone interface. Note that there is very little difference between periods 2 and 10. Again, the sets of ‘on’ cycles are independent events, even though the modulation frequency has increased from 1 kHz to 7.5 kHz.

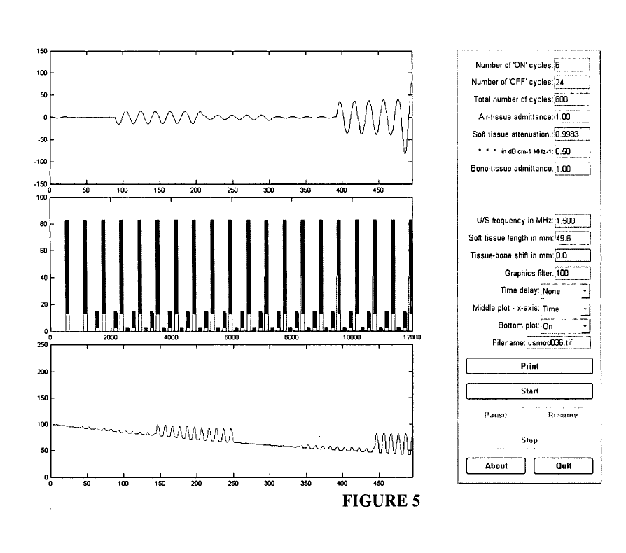

In FIG. 5, the waveform has changed to 6 ‘on’ cycles followed by 24 ‘off’ cycles. The energy and mean intensity are the same and the duty cycle is still 20%.

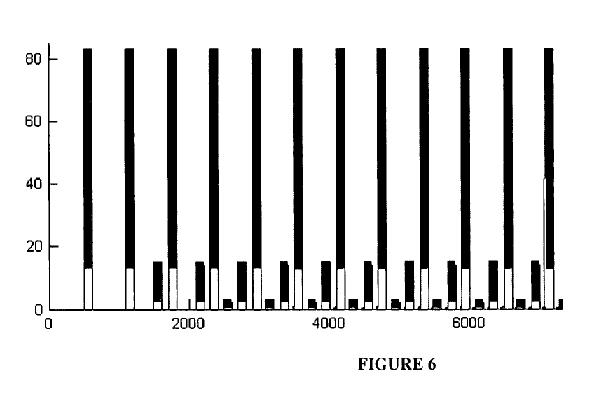

In FIG. 6, the tall bars (height=˜83) are the unreflected cycles reaching the soft-tissue bone interface. The short bars (height=˜16) are the reflected cycles. Note that the second unreflected set of cycles has reached the interface before the reflected set reach the interface. The very short bars (height=˜4) are the re-reflected sets of cycles. Again, all sets of cycles are similar, and it does not matter whether it is the first set of ‘on’ cycles just after the transducer was turned on, or the 100^{th} set.

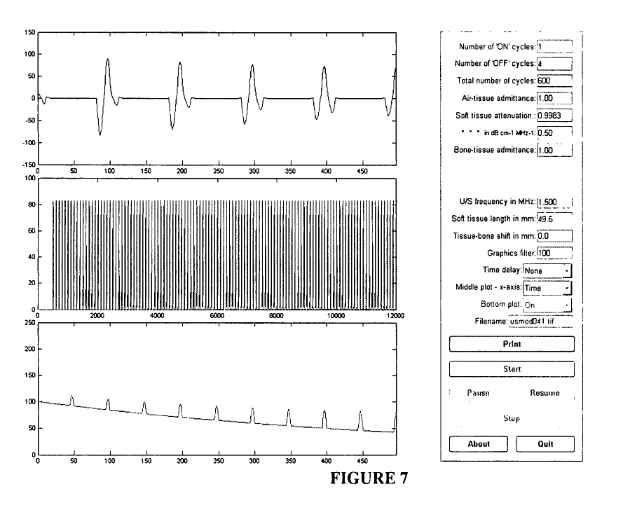

FIG. 7 shows the theoretical maximum modulation for a 20% duty cycle. Clearly, the number of ‘on’ cycles cannot be less than 1, and this fixes the number of ‘off’ cycles to be 4. The modulation frequency is 300 kHz.

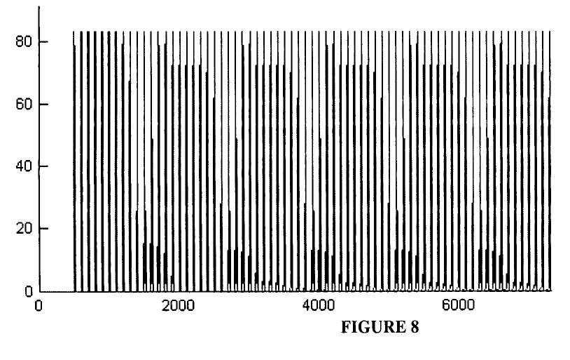

FIG. 8 is an enlarged section of FIG. 7, again showing that all sets of ‘on’ cycles are similar.

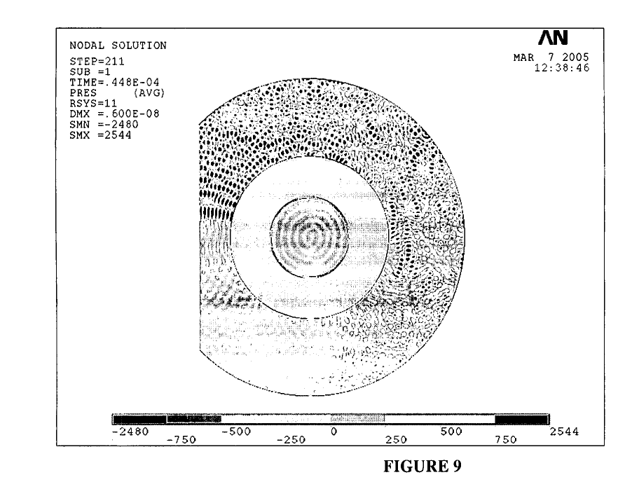

FIG. 9 shows the results of a two-dimensional ultrasound model for an existing Exogen device. The transducer is positioned against the top half of the flat edge of the soft tissue on the left of the plot. The applied pressure range is ±1000 Pa. The figure shows the pressure distribution after 150 cycles of ultrasound. A standing wave can almost be seen in the soft tissue between the transducer and the bone (this is the regular array of very dark regions indicating very low or very high pressure). Note that the pressure distribution in the soft tissue is approximately ±2500 Pa or 2½ times the applied pressure variation. This is due to the multiple interference between two or more cycles that can occur in a two-dimensional model. Clearly, the constructive interference positions are not uniformly distributed.

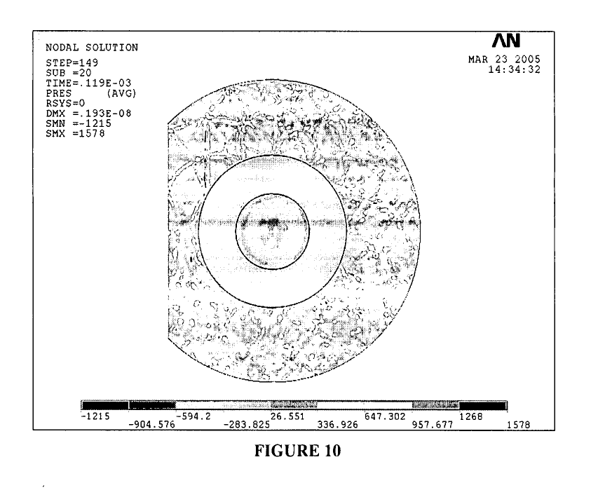

FIG. 10 shows the pressure variation when the modulation frequency is 300 kHz. The applied pressure range is still ±1000 Pa, but the soft tissue pressure range is approximately ¼ to ½ times the applied range. This is about half of the range found in the previous figure. Upon comparison with FIG. 9, it is clear that the constructive interference positions are uniformly distributed.

The following examples provide further information that can be correlated with the Figures as indicated.

### EXAMPLE: 1

| | |
|---|---|
| Title/comments: | An existing Exogen signal |
| Carrier frequency: | 1.5 MHz |
| Modulation frequency: | 1.0 kHz |
| Duty cycle: | 20% |
| Equivalent to: | 300 ‘on’ cycles |
| | 1200 ‘off’ cycles |

### EXAMPLE: 2

| | |
|---|---|
| Title/comments: | The signal in FIGS. 3 and 4 |
| Carrier frequency: | 1.5 MHz |
| Modulation frequency: | 7.5 kHz |
| Duty cycle: | 20% |
| Equivalent to: | 40 ‘on’ cycles |
| | 160 ‘off’ cycles |

### EXAMPLE: 3

| | |
|---|---|
| Title/comments: | The signal in FIGS. 5 and 6 |
| Carrier frequency: | 1.5 MHz |
| Modulation frequency: | 50.0 kHz |
| Duty cycle: | 20% |
| Equivalent to: | 6 ‘on’ cycles |
| | 24 ‘off’ cycles |

### EXAMPLE: 4

| | |
|---|---|
| Title/comments: | The signal in FIGS. 7 and 8, the theoretical |
| | maximum for this carrier frequency |
| Carrier frequency: | 1.5 MHz |
| Modulation frequency: | 300.0 kHz |
| Duty cycle: | 20% |
| Equivalent to: | 1 ‘on’ cycle |
| | 4 ‘off’ cycles |

### EXAMPLE: 5

| | |
|---|---|
| Title/comments: | The maximum frequency of single ‘on’ cycles |
| | for this carrier frequency |
| Carrier frequency: | 1.5 MHz |
| Modulation frequency: | 750.0 kHz |
| Duty cycle: | 50% |
| Equivalent to: | 1 ‘on’ cycle |
| | 1 ‘off’ cycle |

### EXAMPLE: 6

| | |
|---|---|
| Title/comments: | The intensity of the transducer can be |
| | increased as the duty cycle is less. |
| Carrier frequency: | 1.5 MHz |
| Modulation frequency: | 150.0 kHz |
| Duty cycle: | 10% |
| Equivalent to: | 1 ‘on’ cycle |
| | 9 ‘off’ cycles |

### EXAMPLE: 7

| | |
|---|---|
| Title/comments: | The time for the 1000 cycles will equal the |
| | time for the 300 cycles in the existing Exogen |
| | signal as the modulation frequency is the same. |
| Carrier frequency: | 5 MHz |
| Modulation frequency: | 1.0 kHz |
| Duty cycle: | 20% |
| Equivalent to: | 1000 ‘on’ cycles |
| | 4000 ‘off’ cycles |

### EXAMPLE: 8

| | |
|---|---|
| Title/comments: | The theoretical maximum for this carrier |
| | frequency. |
| Carrier frequency: | 5 MHz |
| Modulation frequency: | 1000.0 kHz |
| Duty cycle: | 20% |
| Equivalent to: | 1 ‘on’ cycle |
| | 4 ‘off’ cycles |

### EXAMPLE: 9

| | |
|---|---|
| Title/comments: | The time for the 100 cycles will equal the |
| | time for the 300 cycles in the existing Exogen |
| | signal as the modulation frequency is the same. |
| Carrier frequency: | 0.5 MHz |
| Modulation frequency: | 1.0 kHz |
| Duty cycle: | 20% |
| Equivalent to: | 100 ‘on’ cycles |
| | 400 ‘off’ cycles |

### EXAMPLE: 10

| | |
|---|---|
| Title/comments: | The theoretical maximum for this carrier |
| | frequency. |
| Carrier frequency: | 0.5 MHz |
| Modulation frequency: | 100.0 kHz |
| Duty cycle: | 20% |
| Equivalent to: | 1 ‘on’ cycles |
| | 4 ‘off’ cycles |

Our research has shown that the existing Exogen device is very robust to bone geometry, soft tissue depth, and the placement of the transducer with respect to the fracture. This Exogen device would not provide such a robust technique if it was essential for the ultrasound to travel in a straight line between the transducer and the key cells. The ultrasound leaves the transducer and is reflected inside the soft tissue and bone until it reaches the particular cells that need to be activated in order to lead to osteogenesis. Reflection of the ultrasound creates interference patterns between the initial signal and the signal reflected off the soft tissue-bone and the soft tissue-air interfaces. Constructive interference can cause pressure variations much greater than those caused by the initial signal alone. Similarly, destructive interference can create regions of little pressure variations.

The positions of constructive interference move round within the soft tissue, and can be adjacent to the bone. If these positions of constructive interference move to the cells that need to be activated the healing process is initiated. Surprisingly, it is not the distribution of ultrasound that is important, but the distribution of constructive interference.

Therefore, the present invention improves healing of bone fractures by maximising bone repair as a result of generating a uniform distribution of constructive interference positions in the target site. The present invention also improves healing of bone fractures by maximising bone repair as a result of maximising the density of constructive interference positions in the target site. In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A method for healing bone fractures, comprising applying an ultrasound signal to a target site, wherein the ultrasound signal comprises a generally uniform distribution of constructive interference positions in the target site.

2. (canceled)

3. The method according to claim 1, wherein the signal properties are manipulated in order to increase the density of constructive interference positions in the target site.

4. The method according to claim 2, wherein the ultrasound signal comprises a carrier frequency, a modulation frequency and an intensity.

5. (canceled)

6. The method according to claim 4 further comprising the step of varying the modulation frequency.

7. The method according to claim 6, wherein the modulation frequency is at least 10 kHz.

8. The method according to claim 7, wherein the modulation frequency is in the range 10-1000 kHz.

9-11. (canceled)

12. The method according to claim 8, wherein the modulation frequency is in the range 80-300 kHz.

13. (canceled)

14. The method according to claim 8, wherein the carrier frequency is in the range 20 kHz-10 MHz.

15. The method according to claim 14, wherein the carrier frequency is about 1.5 MHz.

16. The method according to claim 14, wherein the intensity is in the range 50-1000 mWcm⁻².

17. The method according to claim 16, wherein the intensity is about 150 mWcm⁻¹.

18. The method according to claim 1, wherein the ultrasound signal is pulsed.

19. The method according to claim 18, wherein the pulsed ultrasound signal has a duty cycle in the range 5-50%.

20. The method according to claim 19, wherein the duty cycle is 20%.

21. (canceled)

22. An apparatus for healing bone fractures, comprising:
an electro-acoustic transducer for producing an ultrasound signal and configured to transmit the ultrasound signal to a target site;
and a generator means for exciting the transducer with an electrical-output signal, wherein the ultrasound signal comprises a carrier frequency, a modulation frequency and an intensity, the modulation frequency creates a generally uniform distribution of constructive interference positions in the target site.

23. (canceled)

24. The apparatus according to claim 22, wherein the modulation frequency is at least 10 kHz.

25. The apparatus according to claim 24, wherein the modulation frequency is in the range 10-1000 kHz.

26-28. (canceled)

29. The apparatus according to claim 25, wherein the modulation frequency is in the range 80-300 kHz.

30. (canceled)

31. The apparatus according to any of claim 25, wherein the carrier frequency is in the range 20 kHz-10 MHz.

32. The apparatus according to claim 31, wherein the carrier frequency is about 1.5 MHz.

33. The apparatus according to claim 32, wherein the intensity is in the range 50-1000 mWcm⁻².

34. The apparatus according to claim 33, wherein the intensity is about 150 mWcm².

35. The apparatus according to claim 22, wherein the ultrasound signal is pulsed.

36. The apparatus according to claim 35, wherein the pulsed ultrasound signal has a duty cycle in the range 5-50%.

37. The apparatus according to claim 36, wherein the duty cycle is 20%.

38-39. (canceled)
